(19) United States
(12) Patent Application Publication
(10) Pub. No.: US 2004/0058976 A1
(43) Pub. Date: Mar. 25, 2004

(54) Ophthalmic compositions for treating ocular hypertension

(76) Inventor: Christine Stelmach, Westfield, NJ (US)

Correspondence Address: MERCK AND CO INC, P O BOX 2000, RAHWAY, NJ, 070650907

(21) Appl. No.: 10/466,877

(22) PCT Filed: Jan. 25, 2002
(86) PCT No.: PCT/US02/02011

(51) Int. Cl. A61K 38/04; A61K 31/405; A61K 31/21

(52) U.S. Cl. 514/414; 514/419; 514/19; 514/513

(57) ABSTRACT

This invention relates a formulation comprising potent potassium channel blockers or pharmaceutically acceptable salts thereof in combination with peanut oil for the treatment of glaucoma and other conditions which leads to elevated intraoccular pressure in the eye of a patient. This invention also relates to the use of such compounds to provide a neuroprotective effect to the eye of mammalian species, particularly humans.

### BACKGROUND OF THE INVENTION

Recently potassium channel blockers were found to reduce intraocular pressure in the eye and therefore provide an approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Ophthalmol. Vis. Sci 38, 1997; WO 89/10757, WO94/28900, and WO 96/33719).

The present invention relates to potassium channel blocker compositions, and methods of preparation thereof. The composition can be used in the treatment of glaucoma and/or ocular hypertension. The composition provides ease for ocular dosing in solution, and a favorable therapeutic efficacy and duration of action.

Previously compounds which were insoluble in aqueous vehicles were suspended in hydroxy-cellulose for ocular dosing. The disadvantages of ocular dosing of suspensions is that particle size must be controlled for drug dissolution and for patient comfort, and therapeutic effect is dependent on the dissolution rate of drug particles.

The instantly claimed invention relates to a composition containing a potassium channel blocker in combination with peanut oil, which allows compounds which are not soluble in aqueous solution to be dosed in solution thereby avoiding the problems associated with ocular dosing of suspensions. See Yamamoto et al., S.T.P. Pharma Sciences 4(2) 133-138, 1994 for a discussion on the use of peanut oil as a vehicle for dosing of insulin suspensions (incorporated herein by reference).

### SUMMARY OF THE INVENTION

This invention relates to novel ophthalmic compositions comprising potent potassium channel blockers in combination with peanut oil. This invention also relates to novel ophthalmic compositions comprising potent potassium channel blockers of the structural formulas:

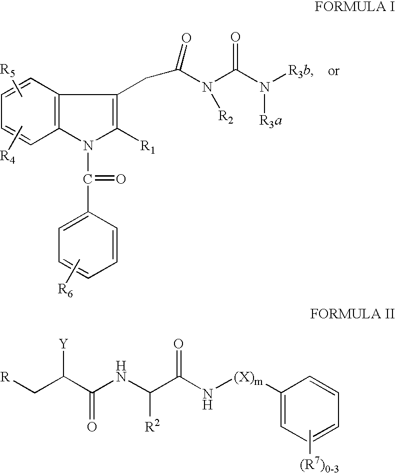

or a pharmaceutically acceptable salt, enantiomer, diastereomer or mixture thereof: wherein,

R and R^{x} independently represent C₁₋₆ alkyl, (CH₂)ₙaryl, (CH₂)ₙheteroaryl, (CH₂)ₙ heterocycloalkyl, said alkyl, aryl or heteroaryl optionally substituted with 1-3 groups of R^{y};

Y represents —(CH₂)ₙSCOR^{z};

X represents CH₂, or O (in which m does not exist);

R^{y} represents hydrogen, C₁₋₆ alkoxy, C₁₋₆ alkyl, CF₃, nitro, amino, cyano, C₁₋₆ alkylamino, or halogen and

R^{z} represents C₁₋₆ alkoxy, or C₁₋₆ alkyl;

m represents 1-3;

n represents 0-3

R₁ represents hydrogen or C₁₋₆ alkyl;

R₂, R₃a and R₃b independently represent hydrogen, C₁₋₁₀ alkyl, C₃₋₁₀ cycloalkyl, C₄₋₁₀ heterocycloalkyl, C₄₋₁₀ heteroaryl, or C₆₋₁₀ aryl;

R₄ represents hydrogen, C₁₋₆ alkoxy, C₁₋₆ alkyl, CF₃, or halogen;

R₅ represents hydrogen, C₁₋₆ alkoxy, C₁₋₆ alkyl, CF₃, nitro, amino, cyano, C₁₋₆ alkylamino, or halogen;

R₆ represents hydrogen, halogen or C₁₋₆ alkyl; and

R⁷ represents H, halo, OH, NO₂, NH₂, CN, alkoxy, —COO—, alkoxycarbonyl, haloalkyl, alkoxycarbonylalkyl, or alkylsulphonyl in combination with peanut oil.

This invention further relates to a composition comprising peanut oil in combination with the potassium channel blockers disclosed in Merck & Co., Inc. Attorney Docket number 20798PV under U.S. Ser. No. 60/264,954, filed simultaneously with this application, U.S. Ser. No. 60/176,695, filed Jan. 18, 2000 and U.S. Ser. No. 60/176,694, filed Jan. 19, 2000 all incorporated herein by reference.

The compositions are useful in the treatment of glaucoma and other conditions which are related to elevated intraocular pressure in the eye of a patient. This invention also relates to the use of such compositions to provide a neuroprotective effect to the eye of mammalian species, particularly humans.

This and other aspects of the invention will be realized upon inspection of the invention as a whole.

### DETAILED DESCRIPTION OF THE INVENTION

The invention is described herein in detail using the terms defined below unless otherwise specified.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, without alternating or resonating double bonds between carbon atoms. It may contain from 1 to 4 rings which are fused.

Alkoxy refers to C₁-C₆ alkyl—O—, with the alkyl group optionally substituted as described herein.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. The preferred aryl groups are phenyl, naphthyl and phenanthrenyl. Aryl groups may likewise be substituted as defined. Preferred substituted aryls include phenyl and naphthyl.

The term “heterocycloalkyl” refers to a cycloalkyl group (nonaromatic) in which one of the carbon atoms in the ring is replaced by a heteroatom selected from O, S or N, and in which up to three additional carbon atoms may be replaced by hetero atoms.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of this type are pyrrole, pyridine, oxazole, thiazole and oxazine. Additional nitrogen atoms may be present together with the first nitrogen and oxygen or sulfur, giving, e.g., thiadiazole.

One embodiment of this invention is realized when R₂ is C₁₋₆ alkyl or C₃₋₁₀ cycloalkyl and all other variables are as originally described for the compounds of formula I.

Another embodiment of this invention is realized when R₃b is C₁₋₆ alkyl or C₃₋₁₀ cycloalkyl and all other variables are as originally described for the compounds of formula I.

A preferred embodiment of this invention is realized when R₁ is C₁₋₆ alkyl, R₂ and R₃b independently are C₁₋₆ alkyl or C₃₋₁₀ cycloalkyl, R₄ is hydrogen, R₅ is C₁₋₆ alkoxy, C₁₋₆ alkyl, R₃a is hydrogen and R₆ is halogen or C₁₋₆ alkyl for the compounds of formula I.

A more preferred embodiment of this invention is realized when R₁ is C₁₋₃ alkyl, R₂ and R₃b independently are C₁₋₄ alkyl or C₅₋₁₀ cycloalkyl, R₄ is hydrogen, R₅ is C₁₋₃ alkoxy, and R₆ is halogen for the compounds of formula I.

One embodiment of this invention is realized when R is C₁₋₆ alkyl, or (CH₂)ₙ aryl, and all other variables are as originally described for the compounds of formula II.

Another embodiment of this invention is realized when R^{x} is C₁₋₆ alkyl, or (CH₂)ₙaryl, and all other variables are as originally described for the compounds of formula II.

Yet another embodiment of this invention is realized when X is CH₂ and all other variables are as originally described for the compounds of formula II.

Still another embodiment of this invention is realized when Y is —(CH₂)ₙSCOR^{z} wherein n=0, and all other variables are as originally described for the compounds of formula II.

Another embodiment of this invention is realized when Y is —(CH₂)ₙSCOR^{z}, wherein n=1-3, and all other variables are as originally described for the compounds of formula II.

A preferred embodiment of this invention is realized when R is (CH₂)ₙ aryl, R^{x} is C₁₋₆ alkyl, Y is (CH₂)ₙSCOR^{z}, X is CH₂ and m=1 for the compounds of formula II.

Another preferred embodiment of this invention is realized when R is (CH₂)ₙaryl, R^{x} is C₁₋₆ alkyl, Y is (CH₂)ₙSCOR^{z}, X is CH₂ and m=2 for the compounds of formula II.

Still another preferred embodiment of this invention is realized when R is C₁₋₆ alkyl, R^{x} is (CH₂)ₙaryl, Y is (CH₂)ₙ SCOR^{z}, X is CH₂ and m=2 for the compounds of formula II.

Yet another preferred embodiment of this invention is realized when R is (CH₂)ₙaryl, R^{x} is (CH₂)ₙaryl, Y is (CH₂)ₙ SCOR^{z}, X is CH₂ and m=2 for the compounds of formula II.

Preferred maxi-K channel blockers of the claimed composition are selected from:

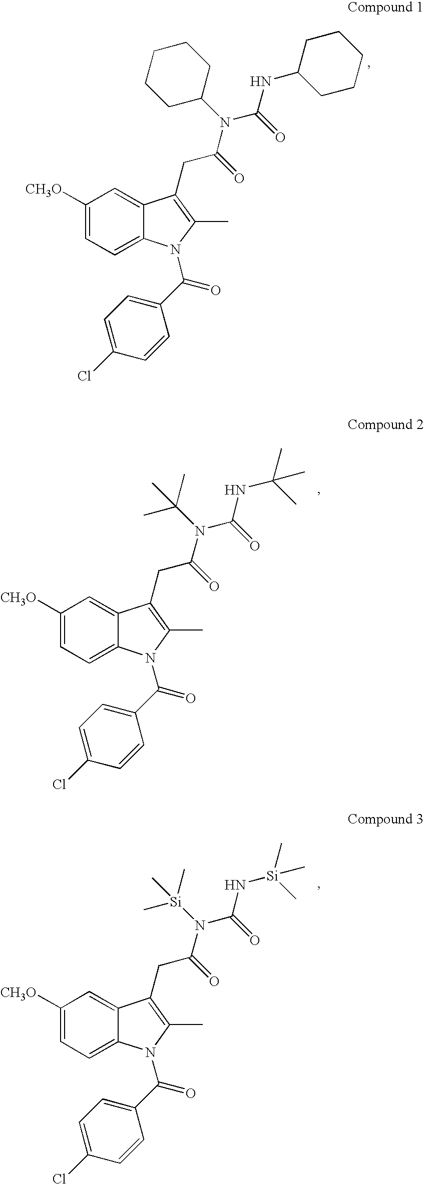

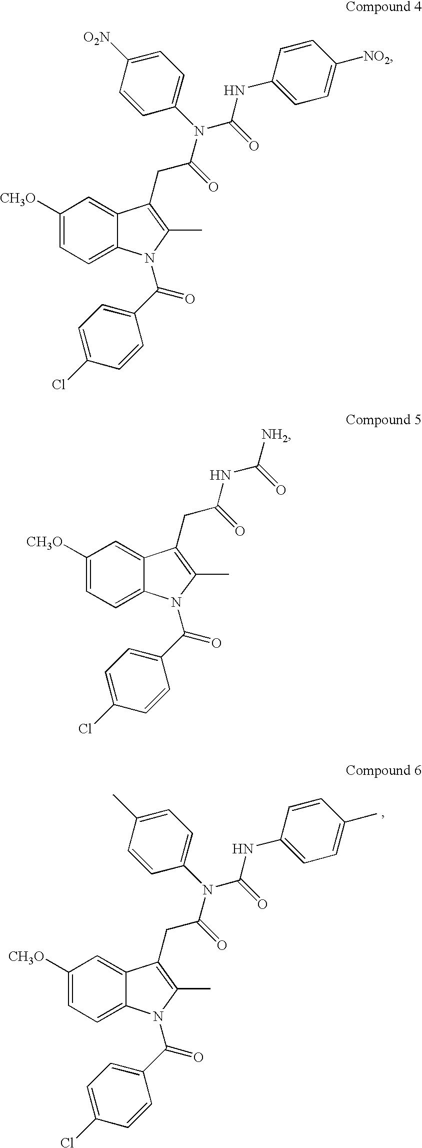

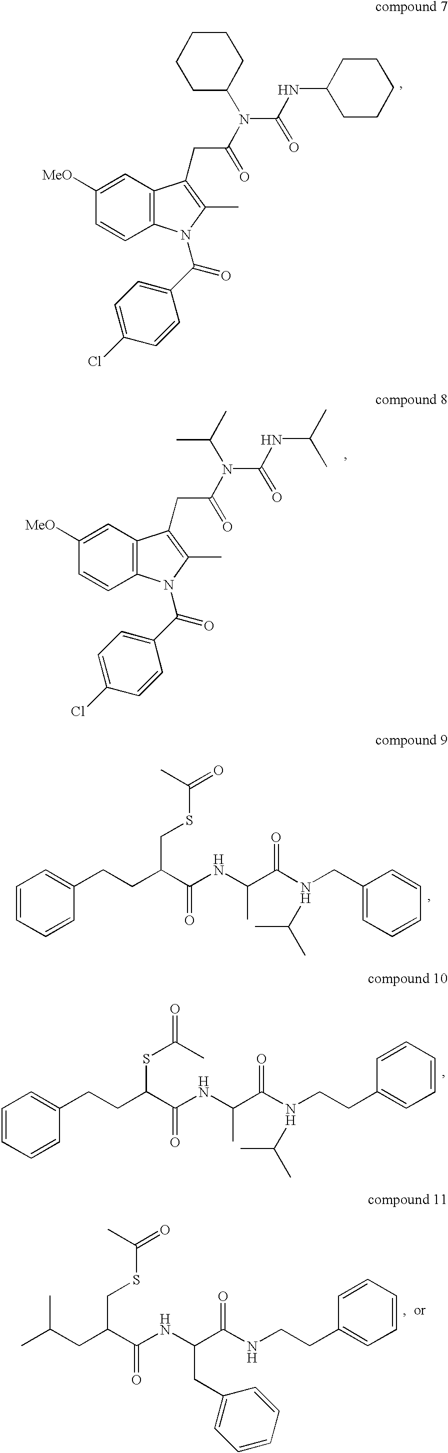

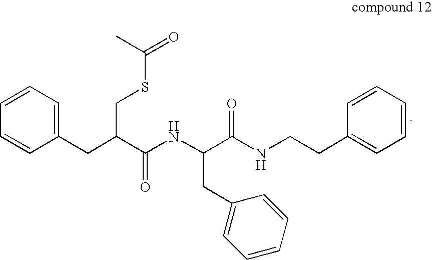

This invention is also concerned with compositions comprising other ocular thereapeutic agents such as penitrem-A, paxillene, β-adrenergic blocking agents (betaxolol, bufenolol, carteolol, levobunolol, metipranolol or timolol or a pharmaceutically acceptable salt thereof), parasympathomimetic agents such as pilocarpine, carbonic anhydrase inhibitors (dorzolamide, acetazolamide, metazolamide or brinzolamide or a pharmaceutically acceptable salt thereof), prostaglandin (latanoprost, rescula, S 1033 or a hypotensive lipid derived from PGF2α prostaglandins such as prostamide (AGN 192024)) either alone or in combination thereof along with peanut oil. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1—OCH₃), or a hydroxy group (PGF₂ₐ 1—OH). One or more of these ocular therapeutic agents can be combined with the potassium channel blockers and peanut oil of the claimed composition.

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

Intraocular pressure (IOP) is controlled by aqueous humor dynamics. Aqueous humor is produced at the level of the non-pigmented ciliary epithelium and is cleared primarily via outflow through the trabecular meshwork. Aqueous humor inflow is controlled by ion transport processes. It is thought that maxi-K channels in non-pigmented ciliary epithelial cells indirectly control chloride secretion by two mechanisms; these channels maintain a hyperpolarized membrane potential (interior negative) which provides a driving force for chloride efflux from the cell, and they also provide a counter ion (K⁺) for chloride ion movement. Water moves passively with KCl allowing production of aqueous humor. Inhibition of maxi-K channels in this tissue would diminish inflow. Maxi-K channels have also been shown to control the contractility of certain smooth muscle tissues, and, in some cases, channel blockers can contract quiescent muscle, or increase the myogenic activity of spontaneously active tissue. Contraction of ciliary muscle would open the trabecular meshwork and stimulate aqueous humor outflow, as occurs with pilocarpine. Therefore maxi-K channels could profoundly influence aqueous humor dynamics in several ways; blocking this channel would decrease IOP by affecting inflow or outflow processes or by a combination of affecting both inflow/outflow processes.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compositions of this invention are useful for treating macular edema and/or macular degeneration.

Macular edema is swelling within the retina within the critically important central visual zone at the posterior pole of the eye. An accumulation of fluid within the retina tends to detach the neural elements from one another and from their local blood supply, creating a dormancy of visual function in the area.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, Apr. 5, 1991, pp. 1593-99. It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, the claimed composition can be useful for increasing retinal and optic nerve head blood velocity, increasing retinal and optic nerve oxygen tension as well as providing a neuroprotective effect or a combination thereof.

The herein examples illustrate but do not limit the claimed invention. The claimed formulations are potassium channel antagonists in combination with peanut oil and are thus useful in the described neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

The maxi-K channel blocker formulations of the instant invention can be administered in a therapeutically effective amount intravaneously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert.

The novel ophthalmic formulations of this invention comprise from 0.01 to 5% wt./wt. maxi-K channel blocker/peanut oil and especially 0.1 to 2% of medicament in peanut oil. Higher dosages as, for example, about 10% or lower dosages can be employed provided the dose is effective in reducing intraocular pressure, treating glaucoma, increasing blood flow velocity or oxygen tension. For a single dose, from between 0.001 to 5.0 mg, preferably 0.005 to 2.0 mg, and especially 0.005 to 1.0 mg of the maxi-K channel blocker in peanut oil can be applied to the human eye.

The novel method of this invention comprises the topical ocular administration of about 0.001 to 5 mg per day, preferably about 0.25 to 3 mg per day, of a maxi-K channel blocker contained in peanut oil to each eye.

Regarding combinations, the ophthalmic formulations of the claimed invention will comprise about, for example, 0.05 to 5% (w/w) of a carbonic anhydrase inhibitor or prostaglandin, usually about 0.5 to 3% (w/w) or about 0.01 to 1% (w/w) of B-adrenergic antagonist, preferably about 0.1 to 0.5% (w/w) in combination with the maxi-K channel blocker and peanut oil, which can be administered on a 1 to 2 times a day schedule. Alternatively, the ophthalmic formulation can contain, for example 0.05 to 5% (w/w) of a carbonic anhydrase inhibitor, 0.01 to 1% (w/w) of β-adrenergic antagonist and 0.01 to 5% (w/w) of a maxi-K channel blocker in combination with 0.01 to 5% peanut oil.

The pharmaceutical preparation which contains the compound may be conveniently admixed with other non-toxic pharmaceutical organic carriers, or with other non-toxic pharmaceutical inorganic carrier. Typical of other pharmaceutically acceptable carriers are, for example, water, mixtures of water and water-miscible solvents such as lower alkanols or aralkanols, vegetable oils, polyalkylene glycols, petroleum based jelly, ethyl cellulose, hydroxypropyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000. The peanut oil vehicle is any preparation containing peanut oil in combination with a maxi-K channel blocker.

Suitable subjects for the administration of the formulation of the present invention include primates, man and other animals, particularly man and domesticated animals such as cats and dogs.

The ophthalmic solution or suspension may be administered as often as necessary to maintain an acceptable IOP level in the eye. It is contemplated that administration to the mamalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The pharmaceutical preparation may also be in the form of a solid insert such as one which after dispensing the drug remains essentially intact as described in U.S. Pat. Nos. 4,256,108; 4,160,452; and 4,265,874; or a bio-erodible insert that either is soluble in lacrimal fluids, or otherwise disintegrates as described in U.S. Pat. No. 4,287,175 or EPO publication 0,077,261.

The following examples of ophthalmic formulations, wherein the active ingredient is dissolved in peanut oil are given by way of illustration.

| | SOLUTION COMPOSITIONS | | | | | |
|---|---|---|---|---|---|---|
| Test | I | II | III | IV | V | VI |
| Formulation 1 | | | | | | |
| Compound 3 | 0.5% | 0.2% | 0.1%, | 0.05% | 0.025% | 0.01% |
| Peanut oil | | | | | | |
| Formulation 2 | | | | | | |
| Timolol-free base | 0.5% | 0.2% | 0.1%, | 0.05% | 0.025% | 0.01% |
| Peanut oil | | | | | | |
| Formulation 3 | | | | | | |
| Paxilline | 0.5% | 0.2% | 0.1%, | 0.05% | 0.025% | 0.01% |
| Peanut oil | | | | | | |
| Formulation 4 | | | | | | |
| Penitrem A | 0.5% | 0.2% | 0.1%, | 0.05% | 0.025% | 0.01% |
| Peanut oil | | | | | | |
| Formulation 5 | | | | | | |
| Compound 2 | 0.5% | 0.2% | 0.1%, | 0.05% | 0.025% | 0.01% |
| Peanut oil | | | | | | |
| Formulation 6 | | | | | | |
| Afletrem | 0.5% | 0.2% | 0.1%, | 0.05% | 0.025% | 0.01% |
| Peanut oil | | | | | | |

The solubility of the formulations are determined using HPLC (Zorbax Rx C8 15 cm×4.6 mm column; 85% acetonitrile/15% 0.1% triethyl ammonium acetate, pH 6.3; flow rate 1 mL/min; wavelength 265 nm; run time 12 minutes).

### EXAMPLE 1

Intraocular pressure measurements (IOP) were performed in conscious adult, pigmented Dutch Belted rabbits (2-4 kg). The animals were allowed free access to standard laboratory diet and tap water, and were housed on a 12 hour light/dark cycle. Seven day rest or washout periods were allowed between assessment of the effects of topically administered treatments on IOP. In order to measure IOP, rabbits were placed in commercially available restraint boxes. Rabbits were conditioned to the restraint boxes on at least two occasions for a period of time ranging up to 6 hours before their first study. On the morning of each experiment, rabbits were placed in restraint boxes for a period of one hour before the first measurement of IOP. IOP was recorded using a pneumatic tonometer (Alcon Applanation Pneumatonograph). Eyelids were gently separated manually and the fingers of the operator were held far enough from the eye to avoid any direct pressure on the eye and surrounding tissues. The membrane of the tonometer was placed on the central part of the cornea. Each measure of IOP lasted 3-5 seconds during which time the animal was completely relaxed. In general, two measures for each eye was sufficient, the first measure being frequently discarded. The tonometer tracing represents the oscillations which correspond to ocular pulsations; the average value of the oscillations corresponded to the level of IOP in mm Hg.

The initial measurement of control IOP was done on a number of rabbits, and 12 rabbits whose control IOP were between 17-25 mm Hg (values of IOP considered to be normal) were selected for study. In each study, the rabbits were separated into two equal subgroups of 6 rabbits each with essentially equivalent control IOPs. Each study consisted of the topical administration of active test substance to one subgroup of 6 rabbits (12 eyes) and vehicle to one subgroup of 6 rabbits (12 eyes); total number of eyes tested per study being 24. In each study, one drop (volume of 50 ul) of active test substance per vehicle was instilled by means of a Hamilton syringe into both eyes of each rabbit at time ) (Tₒ). IOPs were re-measured at 30 min, 1 hour, 2 hours, 3 hours and 4 hours after instillation. Results were expressed as mean+/−SD of changes in IOP (mm Hg) from the basal Tₒ levels measured just prior to the instillation of active test agent or vehicle. The significance of the data was determined as the difference from the Tₒ value using Dunnett's t-test, as well as use of an unpaired Student's t-test for comparisons of changes in IOP between test substance and vehicle at matched time points.

Vehicles consisted of 0.5% aqueous peanut oil. Formulation of test agents in peanut oil was performed as follows: peanut oil formulations were prepared by dissolving 0.5%, 0.2%, 0.1%, 0.05%, 0.025% or 0.01% (wt./wt) of the test compound in peanut oil. Sonnication was used to obtain complete dissolution of the compound.

Table 1 evaluates the effect of 0.5% timolol (free base equivalent) formulated in peanut oil and shows significant IOP lowering with timolol in the peanut oil vehicle. Tables 2 and 3 demonstrate significant IOP lowering with the administration of 0.1% paxilline and penitrm A, respectively (two known maxi-K channel blockers) formulated in peanut oil. Table 4 demonstrates significant IOP lowering with the administration of 0.2% Compound 1 formulated in peanut oil. Table 5 assesses the effects on IOP of 0.1 % afletrem (a known maxi-K channel blocker) formulated in peanut oil.

TABLE 1
Effect of Timolol Free Base (0.5%) in Peanut Oil on IOP in Dutch Belted Rabbits

| Parameter/ | Time | 030 min | 60 min | 120 min | 180 min | 240 min |
|---|---|---|---|---|---|---|
| Comparison | (mm Hg) | (Δmm Hg) | (Δmm Hg) | (Δmm Hg) | (Δmm Hg) | (Δmm Hg) |
| Control Mean | 21.04 | −0.25 | 0.13 | 0.50 | 0 | −0.13 |
| (vehicle) STD | 1.68 | 1.19 | 1.48 | 1.53 | 1.69 | 1.85 |
| P vs Time 0 | | 0.64 | 0.83 | 0.35 | 1.0 | 0.83 |
| Treated Mean | 21.00 | −2.63 | −3.46 | −3.96 | −4.21 | −2.04 |
| STD | 1.93 | 2.18 | 2.19 | 1.97 | 1.91 | 1.60 |
| P vs Control | | 0.0001 | 0.0001 | 0.0001 | 0.0004 | 0.0004 |
| P vs Time 0 | | 0.0003 | 0.0001 | 0.0001 | 0.0004 | 0.0005 |

n = 24

TABLE 2
Effect of Paxilline (0.1%) in Peanut Oil on IOP in Dutch Belted Rabbits

| Parameter/ | Time 0 | 30 min | 60 min | 120 min | 180 min | 240 min |
|---|---|---|---|---|---|---|
| Comparison | (mm Hg) | (Δ mm Hg) | (Δ mm Hg) | (Δ mm Hg) | (Δ mm Hg) | (Δ mm Hg) |
| Control Mean | 22.04 | −0.42 | −0.67 | −1.17 | 0 | −0.67 |
| (vehicle) STD | 1.73 1.69 | 1.69 | 2.24 | 1.67 2.08 | | |
| P vs Time | 0 | 0.44 | 0.22 | 0.06 | 1.0 | 0.26 |
| Treated Mean | 22.21 | −0.79 | −2.67 | −2.67 | −1.71 −1.38 | |
| STD | 1.53 1.61 | 2.12 | 1.81 | 1.33 | 1.76 | |
| P vs Control | 0.44 | 0.0008 | 0.01 | 0.0003 | 0.21 | |
| P vs Time 0 | 0.11 | 0.0001 | 0.0001 | 0.001 | 0.008 | |

n = 24

TABLE 3
Effect of Penitrem A (0.1%) in Peanut Oil on IOP in Dutch Belted Rabbits

| Parameter/ | Time 0 | 30 min | 60 min | 120 min | 180 min | 240 min |
|---|---|---|---|---|---|---|
| Comparison | (mm Hg) | (Δ mm Hg) | (Δ mm Hg) | (Δ mm Hg) | (Δ mm Hg) | (Δ mm Hg) |
| Control Mean | 21.75 | 0.50 | 0.21 | −0.88 | −0.67 | −0.46 |
| (vehicle) STD | 1.51 | 0.98 | 1.18 | 2.09 | 2.01 | 1.89 |
| P vs Time 0 | | 0.24 | 0.64 | 0.14 | 0.28 | 0.35 |
| Treated Mean | 21.75 | −1.33 | −2.08 | −3.91 | −2.96 | −2.46 |
| STD | 1.51 | 1.88 | 1.77 | 1.67 | 1.90 | 1.82 |
| P vs Control | | 0.0002 | 0.0001 | 0.0001 | 0.0002 | 0.0005 |
| P vs Time 0 | | 0.02 | 0.0002 | 0.0001 | 0.0001 | 0.0001 |

n = 24

TABLE 4
Effect of Compound 1 (0.2%) in Peanut Oil on IOP in Dutch Belted Rabbits

| Parameter/ | Time | 030 min | 60 min | 120 min | 180 min | 240 min |
|---|---|---|---|---|---|---|
| Comparison | (mm Hg) | (Δmm Hg) | (Δmm Hg) | (Δmm Hg) | (Δmm Hg) | (Δmm Hg) |
| Control Mean | 20.50 | 0.17 | 0.50 | −0.29 | 0.17 | −0.13 |
| (vehicle) STD | 1.53 | 1.55 | 1.84 | 1.37 | 1.66 | 1.75 |
| P vs Time 0 | | 0.73 | 0.33 | 0.57 | 0.76 | 0.82 |
| Treated Mean | 20.50 | −2.0 | −2.58 | −2.96 | −2.42 | −1.58 |
| STD | 1.35 | 1.29 | 0.93 | 1.60 | 1.86 | 2.39 |
| P vs Control | | 0.0001 | 0.0001 | 0.0001 | 0.0001 | 0.02 |
| P vs Time 0 | | 0.0002 | 0.0001 | 0.0001 | 0.0001 | 0.01 |

n = 24

TABLE 5
Effect of Afletrem (0.1%) in Peanut Oil on IOP in Dutch Belted Rabbits

| Parameter/ | Time | 030 min | 60 min | 120 min | 180 min | 240 min |
|---|---|---|---|---|---|---|
| Comparison | (mm Hg) | (Δmm Hg) | (Δmm Hg) | (Δmm Hg) | (Δmm Hg) | (Δmm Hg) |
| Control Mean | 21.83 | 0.17 | 0.88 | 0.04 | 0.63 | 0.38 |
| (vehicle) STD | 1.52 | 1.17 | 1.51 | 1.37 | 1.38 | 1.81 |
| P vs Time 0 | | 0.68 | 0.07 | 0.93 | 0.17 | 0.36 |
| Treated Mean | 21.83 | −1.54 | −2.04 | −2.54 | −2.13 | −1.42 |
| STD | 1.86 | 1.96 | 2.31 | 2.30 | 1.65 | 2.59 |
| P vs Control | | 0.0007 | 0.0001 | 0.0001 | 0.0001 | 0.008 |
| P vs Time 0 | | 0.004 | 0.0004 | 0.0001 | 0.0002 | 0.02 |

n = 24

### Functional Assays

### A. Maxi-k Channel

The identification of inhibitors of the Maxi-K channel is accomplished using Aurora Biosciences technology, and is based on the ability of expressed Maxi-K channels to set cellular resting potential after transient transfection of both α and β 1 subunits of the channel in TsA-201 cells. In the absence of inhibitors, cells display a hyperpolarized membrane potential, negative inside, close to E_{K} (−80 mV) which is a consequence of the activity of the Maxi-K channel. Blockade of the Maxi-K channel will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)). Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization.

Transient transfection of the Maxi-K channel in TsA-201 cells was carried out as previously described (Hanner et al. (1998) J. Biol. Chem. 273, 16289-16296) using FUGENE as the transfection reagent. Twenty-four hours after transfection, cells are collected in Ca²⁺—Mg²⁺-free Dulbecco's phosphate-buffered saline (D-PBS), subjected to centrifugation, plated onto 96-well poly-d-lysine coated plates at a density of 50,000 cells/well, and incubated overnight. The cells are then washed 1× with D-PBS, and loaded with 100 μl of 4 mM CC₂DMPE-0.02% pluronic-127 in D-PBS. Cells are incubated at room temperature for 30 min in the dark. Afterwards, cells are washed 2× with D-PBS and loaded with 100 μl of 5 mM DiSBAC₂(3) in (mM): 140 NaCl, 0.1 KCl, 1 CaCl₂, 0.5 MgCl₂, 20 Hepes-Tris, pH 7.4, 10 glucose. Test compounds are diluted into this solution, and added at the same time. Cells are incubated at room temperature for 30 min in the dark.

Plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 1 CaCl₂, 0.5 MgCl₂, 20 Hepes-Tris, pH 7.4, 10 glucose are added and the fluorescence emission of both dyes recorded for an additional 10 sec. The ratio CC₂DMPE/DiSBAC₂(3), before addition of high-potassium solution equals 1. In the absence of any inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀'s in the range of about 1 nM to about 1 μM, more preferably from about 10 nM to about 200 nM.

### B. Electrophysiological Assays of Compound Effects on High-conductance Calcium-activated Potassium Channels

Human Non-pigmented Ciliary Epithelial Cells

The activity of high-conductance calcium-activated potassium (maxi-K) channels in human non-pigmented ciliary epithelial cells was determined using electrophysiological methods. Currents through maxi-K channels were recorded in the inside-out configuration of the patch clamp technique, where the pipette solution faces the extracellular side of the channel and the bath solution faces the intracellular side. Excised patches contained one to about fifty maxi-K channels. Maxi-K channels were identified by their large single channel conductance (250-300 pS), and by sensitivity of channel gating to membrane potential and intracellular calcium concentration. Membrane currents were recorded using standard electrophysiological techniques. Glass pipettes (Garner 7052) were pulled in two stages with a Kopf puller (model 750), and electrode resistance was 1-3 megohms when filled with saline. Membrane currents were recorded with EPC9 (HEKA Instruments) or Axopatch 1D (Axon Instruments) amplifiers, and digital conversion was done with ITC-16 interfaces (Instrutech Corp). Pipettes were filled with (mM); 150 KCl, 10 Hepes, 1 MgCl₂, 0.01 CaCl₂, 3.65 KOH, pH 7.20. The bath (intracellular) solution was identical, except, in some cases, calcium was removed, 1 mM EGTA was added and 20 mM KCl was replaced with 20 mM KF to eliminate calcium to test for calcium sensitivity of channel gating. Drugs were applied to the intracellular side of the channel by bath perfusion.

Human non-pigmented ciliary epithelial cells were grown in tissue culture as described (Martin-Vasallo, P., Ghosh, S., and Coca-Prados, M., 1989, J. Cell. Physiol. 141, 243-252), and plated onto glass cover slips prior to use. High resistance seals (>1 Gohm) were formed between the pipette and cell surface, and inside out patches were excised. Maxi-K channels in the patch were identified by their gating properties; channel open probability increased in response to membrane depolarization and elevated intracellular calcium. In patches used for pharmacological analysis, removing intracellular calcium eliminated voltage-gated currents. Maxi-K currents were measured after depolarizing voltage steps or ramps that caused channel opening.

The compounds of this invention were applied to the intracellular side of the channel in appropriate concentrations (0.001 to 10 μM). The compounds reduced channel open probability, and this effect was reversed upon washout of compounds from the experimental chamber. The IC50 for block of maxi-K channels under these conditions for the compounds of this invention ranged from about 0.5 nM to about 300 nM.

What is claimed is:

1. An ophthalmic formulation for the treatment of ocular hypertension or gluacoma in a subject in need thereof comprising a potassium channel blocker in combination with a pharmaceutically acceptable peanut oil vehicle.

2. An ophthalmic formulation for the treatment of ocular hypertension or gluacoma in a subject in need thereof comprising 0.01 to 5% (wt/wt) of a potassium channel blocker of the structural formulas: 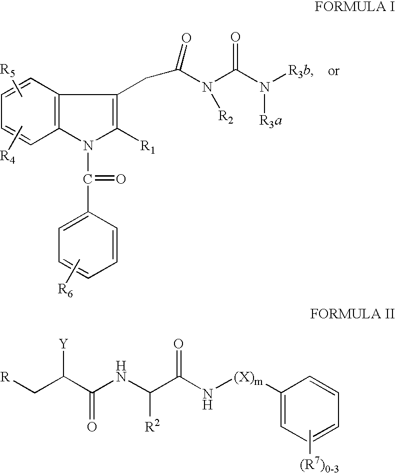
or a pharmaceutically acceptable salt, enantiomer, diastereomer or mixture thereof:
wherein,
R and R^{x} independently represent C₁₋₆ alkyl, (CH₂)ₙaryl, (CH₂)ₙheteroaryl, (CH₂)ₙ heterocycloalkyl, said alkyl, aryl or heteroaryl optionally substituted with 1-3 groups of R^{y};
Y represents —(CH₂)ₙSCOR^{z};
X represents CH₂, or O (in which m does not exist);
R^{y} represents hydrogen, C₁₋₆ alkoxy, C₁₋₆ alkyl, CF₃, nitro, amino, cyano, C₁₋₆ alkylamino, or halogen and
R^{z} represents C₁₋₆ alkoxy, or C₁₋₆ alkyl;
m represents 1-3;
n represents 0-3
R₁ represents hydrogen or C₁₋₆ alkyl;
R₂, R₃a and R₃b independently represent hydrogen, C₁₋₁₀ alkyl, C₃₋₁₀ cycloalkyl, C₄₋₁₀ heterocycloalkyl, C₄₋₁₀ heteroaryl, or C₆₋₁₀ aryl;
R₄ represents hydrogen, C₁₋₆ alkoxy, C₁₋₆ alkyl, CF₃, or halogen;
R₅ represents hydrogen, C₁₋₆ alkoxy, C₁₋₆ alkyl, CF₃, nitro, amino, cyano, C₁₋₆ alkylamino, or halogen;
R₆ represents hydrogen, halogen or C₁₋₆ alkyl; and
R⁷ represents H, halo, OH, NO₂, NH₂, CN, alkoxy, —COO—, alkoxycarbonyl, haloalkyl, alkoxycarbonylalkyl, or alkylsulphonyl, in combination with a pharmaceutically acceptable peanut oil vehicle.

3. The formulation of claim 2 wherein the concentration of maxi-K channel blocker is 0.2 to 2%.

4. The formulation of claim 2 wherein the maxi-K channel blocker is a compound presented by formula I: 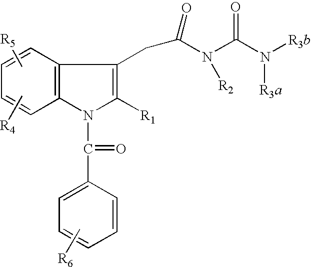
wherein:
R₂ and R₃b independently represent C₁₋₆ alkyl or C₃₋₁₀ cycloalkyl and all other variables are as originally described for the compounds of formula I.

5. A formulation according to claim 4 wherein R₁ is C₁₋₆ alkyl, R₂ and R₃b independently are C₁₋₆ alkyl or C₃₋₁₀ cycloalkyl, R₄ is hydrogen, R₅ is C₁₋₆ alkoxy, or C₁₋₆ alkyl, R₃a is hydrogen and R₆ is halogen or C₁₋₆ alkyl.

6. A formulation according to claim 5 wherein R₁ is C₁₋₃ alkyl, R₂ and R₃b independently are C₁₋₄ alkyl or C₅₋₁₀ cycloalkyl, R₄ is hydrogen, R₅ is C₁₋₃ alkoxy, and R₆ is halogen.

7. The formulation of claim 2 wherein the maxi-K channel blocker is a compound presented by formula II: 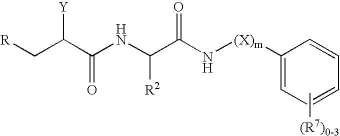
wherein
R and R^{x} independently are C₁₋₆ alkyl, or (CH₂)ₙaryl, X is CH₂, R⁷ is H and Y is —(CH₂)ₙSCOR^{z} wherein n=0, and all other variables are as originally described for the compounds of formula II.

8. A formulation according to claim 2 wherein Y is —(CH₂)ₙSCOR^{z}, wherein n=1-3, and all other variables are as originally described for the compounds of formula II.

9. A formulation according to claim 2 wherein R is (CH₂)ₙ aryl, R^{x} is C₁₋₆ alkyl, Y is (CH₂)ₙSCOR^{z}, X is CH₂ and m=1 for the compounds of formula II.

10. A formulation according to claim 2 wherein R is (CH₂)ₙaryl, R^{x} is C₁₋₆ alkyl, Y is (CH₂)ₙSCOR^{z}, X is CH₂ and m=2 for the compounds of formula II.

11. A formulation according to claim 2 wherein R is C₁₋₆ alkyl, R^{x} is (CH₂)ₙaryl, Y is (CH₂)ₙ SCOR^{z}, X is CH₂ and m=2 for the compounds of formula II.

12. A formulation according to claim 2 wherein R is (CH₂)ₙaryl, R^{x} is (CH₂)ₙaryl, Y is (CH₂)ₙ SCOR^{z}, X is CH₂ and m=2 for the compounds of formula II.

13. A formulation according to claim 2 wherein the maxi-K channel blocker is:

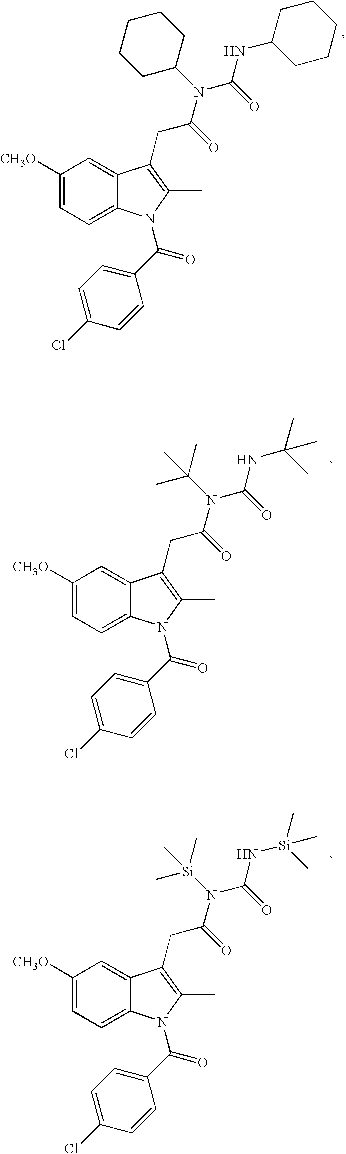

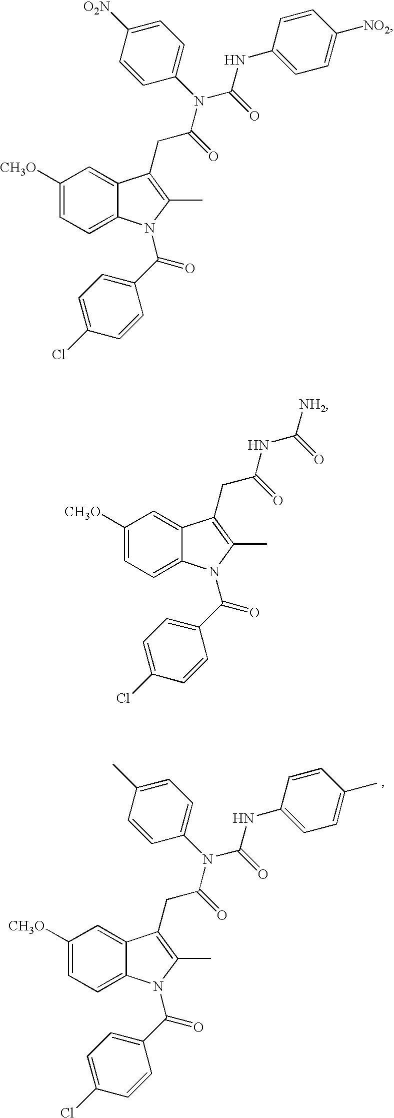

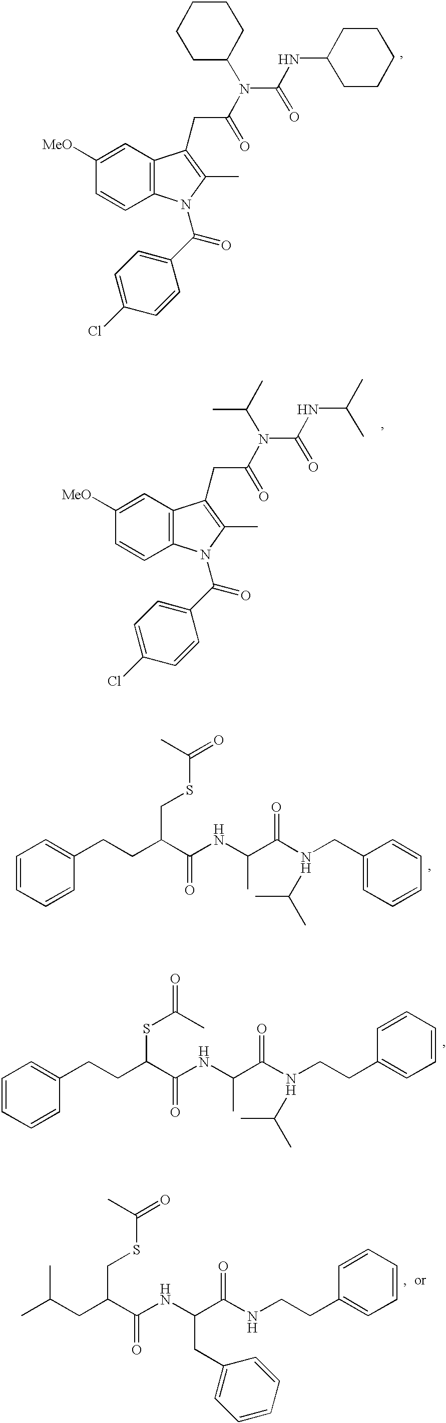

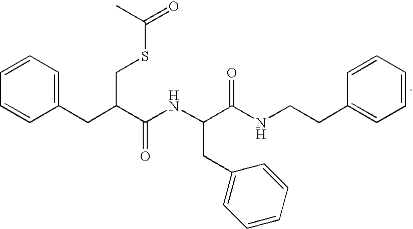

14. A formulation according to claim 2 which also contains 0.01 to 1% of a β-adrenergic antagonist.

15. A formulation according to claim 14 wherein the β-adrenergic antagonist is betaxolol, bufenolol, carteolol, levobunolol, metipranolol, timolol or a pharmaceutically acceptable salt thereof.

16. A formulation according to claim 15 wherein the β-adrenergic antagonist is timolol.

17. A formulation according to claim 2 which also contains 0.05 to 5% of a carbonic anhydrase inhibitor.

18. A formulation according to claim 17 wherein the carbonic anhydrase inhibitor is dorzolamide, brinzolamide or a pharmaceutically acceptable salt thereof.

19. A formulation according to claim 16 which also contains a carbonic anhydrase inhibitor.

20. A formulation according to claim 19 wherein the carbonic anhydrase inhibitor is dorzolamide or brinzolamide.

21. A formulation according to claim 2 which also contains 0.05 to 5% (wt/wt) of a prostaglandin.

22. A formulation according to claim 21 wherein the prostaglandin is latanoprost, rescula, S1033 or a hypotensive lipid derived from PGF2α prostaglandins such as prostamide or a pharmaceutically acceptable salt thereof.

23. A formulation according to claim 1 which is in the form of an ocular insert.
